(19) United States
(12) Patent Application Publication
(10) Pub. No.: US 2025/0138029 A1
(43) Pub. Date: May 1, 2025

(54) METHODS FOR ASSESSING LIKELIHOOD OF POST-OPERATIVE DELIRIUM IN PATIENTS UNDERGOING SURGERY

(71) Applicant: The General Hospital Corporation, Boston, MA (US)

(72) Inventors: Zhongcong Xie, Andover, MA (US); Feng Liang, Charlestown, MA (US)

(21) Appl. No.: 18/834,921

(22) PCT Filed: Feb. 3, 2023
(86) PCT No.: PCT/US2023/061977

§ 371 Date: Jul. 31, 2024

Related U.S. Application Data
(60) Provisional application No. 63/306,126, filed on Feb. 3, 2022.

(51) Int. Cl. G01N 33/68

(52) U.S. Cl. CPC G01N 33/6896; G01N 2440/14; G01N 2800/2814

(57) ABSTRACT

Described herein are methods comprising using measurements of perioperative plasma concentrations of Tau-PT217 and/or Tau-PT181 to predict which subjects are likely to develop postoperative delirium, and to thereby identify which patients are most likely to benefit from interventions to reduce risk or mitigate the severity or consequences of postoperative delirium.

### CLAIM OF PRIORITY

This application claims the benefit of U.S. Provisional Application Ser. No. 63/306,126, filed on Feb. 3, 2022. The entire contents of the foregoing are incorporated herein by reference.

### FEDERALLY SPONSORED RESEARCH OR DEVELOPMENT

This invention was made with Government support under Grant Nos. HD098754, AG062509, and AG070761 awarded by the National Institutes of Health. The Government has certain rights in the invention.

### TECHNICAL FIELD

Described herein are methods comprising using measurements of perioperative plasma concentrations of Tau-PT217 and/or Tau-PT181 to predict which subjects are likely to develop postoperative delirium, and to thereby identify which patients are most likely to benefit from interventions to reduce risk or mitigate the severity or consequences of postoperative delirium.

### BACKGROUND

Postoperative delirium (POD), one of the most common postoperative complications in older patients¹, is associated with nosocomial complications², extended hospital stays³, a higher chance of institutional discharge^{4,5}, and increased morbidity⁵⁻⁸ and mortality^{9,10}. POD can present as a hyperactive form of delirium, with subjects showing restlessness, agitation, hallucinations, and delusions, or a hypoactive form of delirium, characterized by reduced movements, paucity of speech, and unresponsiveness. POD usually peaks between one and three days after a surgical operation.⁴⁷ Because POD can (and often does) occur in subjects who do not have apparent pre-existing delirium or dementia, it is difficult to identify subjects who are likely to experience POD. The annual health care costs in the United States attributable to postoperative delirium are $32.9 billion¹¹.

### SUMMARY

As shown herein, preoperative plasma concentrations of Tau-PT217 and Tau-PT181 are associated with postoperative delirium, with Tau-PT217 being a stronger indicator of postoperative delirium than Tau-PT181. Thus, measurements of perioperative plasma concentrations of Tau-PT217 and/or Tau-PT181 can be used to predict which subjects are likely to develop postoperative delirium, and to thereby identify which patients are most likely to benefit from interventions to reduce risk or mitigate the severity or consequences of postoperative delirium.

Provided herein are methods of determining risk of developing postoperative delirium (POD) in a subject, preferably wherein the subject is a human over the age of 60 or 65. The methods comprise optionally obtaining a sample comprising plasma from the subject; determining a level of Tau isoforms phosphorylated at threonine 217 (Tau-PT217) and/or a level of Tau isoforms phosphorylated at threonine 181 (Tau-PT181) in the sample; comparing the levels of Tau-PT217 and/or Tau-PT181 in the sample to reference levels of Tau-PT217 and/or Tau-PT181; and identifying a subject who has a level of Tau-PT217 and/or Tau-PT181 above the reference level as being at risk of developing postoperative delirium (POD).

In some embodiments, the subject has no clinical signs of Alzheimer's disease (AD), AD Related Dementias (ADRD), and delirium.

In some embodiments, the subject has a preoperative Mini-Mental State Examination (MMSE) score more than 24.

In some embodiments, determining levels of Tau-PT217 and/or Tau-PT181 in the sample comprises using an ultrasensitive assay. In some embodiments, the ultrasensitive assay comprises a nanoneedle-based assay, Single-Molecule Arrays (SIMOA); Molecular On-bead Signal Amplification for Individual Counting (MOSAIC); Meso Scale Discovery (MSD); Single-Molecule Counting (SMC); nucleic acid linked immune-sandwich assay (NULISA); LUMINEX; SOMAscan Assays; mass spectrometry (e.g., MALDI-MS), and/or mass cytometry (e.g., CyTOF).

Also provided herein are methods for reducing risk of developing postoperative delirium (POD) in a subject. The methods comprise optionally obtaining a sample comprising plasma from the subject; determining a level of Tau isoforms phosphorylated at threonine 217 (Tau-PT217) and/or a level of Tau isoforms phosphorylated at threonine 181 (Tau-PT181) in the sample; comparing the levels of Tau-PT217 and/or Tau-PT181 in the sample to reference levels of Tau-PT217 and/or Tau-PT181; and identifying a subject who has a level of Tau-PT217 and/or Tau-PT181 above the reference level as being at risk of developing POD; and administering an intervention to reduce the risk of developing POD to the subject.

In some embodiments, the subject has no clinical signs of Alzheimer's disease (AD), AD Related Dementias (ADRD), and delirium.

In some embodiments, the subject has a preoperative Mini-Mental State Examination (MMSE) score more than 24.

In some embodiments, determining levels of Tau-PT217 and/or Tau-PT181 in the sample comprises using an ultrasensitive assay. In some embodiments, the ultrasensitive assay comprises a nanoneedle-based assay, Single-Molecule Arrays (SIMOA); Molecular On-bead Signal Amplification for Individual Counting (MOSAIC); Meso Scale Discovery (MSD); Single-Molecule Counting (SMC); nucleic acid linked immune-sandwich assay (NULISA); LUMINEX; SOMAscan Assays; mass spectrometry (e.g., MALDI-MS), and/or mass cytometry (e.g., CyTOF).

In some embodiments, the intervention comprises avoiding the use of antihistamines, anticholinergics, tricyclic antidepressants, benzodiazepines, muscle relaxants, meperidine, gabapentinoids, and scopolamine; and avoiding perioperative polypharmacy.

In some embodiments, the intervention comprises administration of prophylactic low-dose antipsychotics, optionally olanzapine, quetiapine, or risperidone, can be included.

In some embodiments, the intervention comprises sedation using alpha-2 agonists, optionally clonidine or dexmedotomidine.

In some embodiments, the intervention comprises administering ketamine during anesthetic induction; use of regional and neuraxial anaesthesia; xenon anesthesia; and/or administration of dexmedotomidine.

In some embodiments, the intervention comprises pre- and post-operative behavioral interventions and additional care measures, optionally following comprehensive geriatric assessment (CGA)-based perioperative care, ABCDE bundle, or Hospital Elder Life Program (HELP) or modified HELP.

In some embodiments, the behavioral interventions and additional care measures comprise deliberately orienting the patient to place, time and reason for hospitalization; encouraging mobilization with at least daily walks; maintaining hydration (avoiding prolonged (>6 h) fluid fasting); avoiding sleep deprivation; and reminding patients to use their glasses and hearing aids when appropriate.

In some embodiments, the intervention comprises pharmacologic interventions, optionally selected from pre- and post-operative pain management (optionally with opioid-sparing analgesia); administration of NSAIDs and paracetamol/acetaminophen; administration of melatonin receptor agonists such as melatonin and ramelteon; and administration of synthetic corticosteroids such as dexamethasone.

Also described herein are methods of assessing the risk of a patient having post-operative delirium comprising (a) drawing a sample of blood from the patient pre-operatively; and (b) measuring the pre-operative amount of Tau-PT217 and/or Tau-PT181 in the blood plasma, wherein the higher the amount of either or both Tau-PT217 or TauPT181 in the plasma, the more likely the patient will have delirium post-operatively and (c) in those patients in which post-operative delirium is likely, administering an appropriate postoperative therapy to the patient. The identified high-risk patients will have specific interventions of postoperative delirium, including HELP program, ABCDE bundle treatment and others.

In some embodiments, if the blood concentration of either Tau-PT217 or Tau-PT181 is high (Tau-PT217 >75^{th} percentile arbitrary unit [a.u.] or 2.5 pg/ml¹⁵ or Tau-PT181 >75^{th} percentile a.u. or 8 μg/ml¹⁶), the patient is likely to have delirium post-operatively. In some embodiments, the measuring step is carried out using a nanoneedle array as described herein.

Also provided herein are methods of determining post-operatively if a patient is suffering from post-operative delirium comprising (a) drawing a sample of blood from the patient post-operatively; and (b) measuring the amount of Tau-PT217 and/or Tau-PT181 in the blood plasma, wherein the higher the amount of either or both Tau-PT217 or TauPT181 in the plasma indicates that the patient has delirium, in which case the patient should be treated with an appropriate intervention.

In some embodiments, the appropriate intervention or post-operative therapy is selected from HELP, ABCDE bundle, or a pharmacologic intervention or therapy.

Further provided herein are any and all compositions, articles of manufacture, methods, and uses disclosed and/or described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Flow diagram. The flow diagram shows that 491 participants were screened for the studies, and 220 were initially enrolled. Eighty-one participants were excluded, resulting in 139 participants for the final data analysis.

FIGS. 2A-B: Different preoperative plasma concentrations of Tau-PT217 and Tau-PT181 between the participants with postoperative delirium and those without postoperative delirium. Participants who developed postoperative delirium (N=18) had higher preoperative plasma concentrations of Tau-PT217 (A) and Tau-PT181 (B) than the participants who did not develop postoperative delirium (N=121). The student's t-test was used to analyze the data presented in FIGS. 2A and 2B. The P values refer to the differences in the preoperative plasma concentrations of Tau-PT217 or Tau-PT181 between the participants with postoperative delirium and the participants without postoperative delirium. Error bar indicates standard deviation. Tau-PT217, Tau phosphorylation at threonine 217; Tau-PT181, Tau phosphorylation at threonine 181; a.u., arbitrary unit.

FIG. 3. The sensitivity, specificity, and AUC (Area Under The Curve) ROC (Receiver Operating Characteristics) curve of preoperative plasma concentration of Tau-PT217 and Tau-PT181 in predicting postoperative delirium. Tau-PT217 reported a higher discriminatory ability than Tau-PT181. This can be observed in the AUC, or area under the ROC curve.

FIGS. 4A-B. Peptide arrays validate the specificity of Tau-PT217 and Tau-PT181 antibodies. A. Scheme of Tau (2N4R) with phosphorylation sites tested in this study. pT: phospho-threonine, pS: phospho-serine. B. Synthetic peptides (SEQ ID NOs. 1-12, respectively) at different phosphorylation sites are tested against the anti-Tau-PT217 antibody or anti-Tau-PT181 antibody used in the current study. Peptides of 100, 50, 25, and 0 ng were spotted on a nitrogenous membrane and measured with 0.5 mg/ml anti-Tau-PT217 or anti-Tau-PT181. Titrated signals were observed for both antibodies to their specific residues. No cross-reactive signals were observed to other residues on the dot blot.

FIGS. 5A-F. Comparison of Nanoneedle technology with ELISA methods in human SY5Y cell lysates. A-D. Human SH-SY5Y cells were treated with 3% anesthetic sevoflurane for 0, 3, 6, 9 and 12 hours to induce the pathogenesis of Tau-PT217 and Tau-PT181. Tau-PT217 (A) and Tau-PT181 (B) were measured with ELISA kits using 50 μL sample volume. Tau-PT217 (C) and Tau-PT181 (D) were further diluted at 10× into the cell extraction buffer and measured with the Nanoneedle technology using 10 μL sample volume. The dilution was to place the Nanoneedle measurement in the similar detection range (relative concentration with arbitrary unit) as in the plasma samples in the study. E, F. Correlation of the measurement results from ELISA kits and Nanoneedle technology for Tau-PT217 (E) and Tau-PT181 (F). The Pearson's correlation coefficients between Nanoneedle and ELISA measurement were 0.88 and 0.83 for Tau-PT217 and Tau-PT181, respectively. These data indicated that Nanoneedle methods and ELISA methods were well correlated.

### DETAILED DESCRIPTION

Population studies have demonstrated a strong bidirectional association between Alzheimer's disease (AD), AD Related Dementias (ADRD), and delirium¹². Specifically, patients with underlying ADRD are 2.5 to 4.7 times more likely to develop delirium, and patients with delirium face a 12.5-fold increased incidence of newly diagnosed ADRD^{4,13-15} The underlying pathogenic basis of this association, however, remains unclear, and POD can occur in subjects with no clinical evidence of AD, ADRD, or delirium.

Previous studies have shown that a higher preoperative cerebrospinal fluid (CSF) Tau/β-amyloid ratio was associated with higher incidence and greater severity of postoperative delirium¹⁶. These results suggest the potential association of AD neuropathogenesis with postoperative delirium. However, in our prior work, we only measured Tau, not phosphorylated Tau, in CSF and not in plasma. Given the challenges of obtaining CSF in older surgical patients and that plasma biomarkers are efficient with low cost to perform delirium clinical research, we now examine newly emerging blood-based biomarkers of AD and their associations with delirium.

Tauopathy is one of the hallmarks of AD neuropathogenesis^{17,18}. Tau phosphorylation at threonine 217 (Tau-PT217) and 181 (Tau-PT181) are newly identified AD plasma biomarkers¹⁹⁻²⁷. Specifically, a recent clinical investigation has shown that plasma Tau-PT181 can differentiate AD from other neurodegenerative disorders²³. Plasma Tau-PT217 levels increase during the early preclinical state of AD patients when insoluble Tau aggregates are not yet detectable by Tau-positron emission tomography²⁶.

Plasma concentrations of interleukin 6²⁸, c-reactive protein²⁹, neurofilament light³⁰, chitinase-3-like protein 1³¹, and metabolites³² have been shown to associate with postoperative delirium. Moreover, a recent study showed that the changes between the preoperative plasma Tau concentration and the postoperative day 1 plasma Tau concentration were greater in patients who developed postoperative delirium and were associated with delirium severity. Plasma Tau concentrations also predicted the recovery from postoperative delirium³³. However, the association between preoperative plasma concentrations of Tau-PT217 or Tau-PT181 and postoperative delirium has not been determined.

The present prospective observational cohort study was performed to determine the association between Tau-PT217 or Tau-PT181 and the presence or severity of postoperative delirium in patients who had surgery under general or spinal anesthesia. It was hypothesized that elevated preoperative plasma concentrations of Tau-PT217 and Tau-PT181 would be associated with an increased presence and severity of postoperative delirium in patients. The results, shown herein, revealed that patients with higher preoperative plasma concentrations of Tau-PT217 or Tau-PT181 were more likely to experience delirium and also had higher postoperative delirium severity. These data suggest that Tau phosphorylation, part of the AD neuropathogenesis, contributes, at least partially, to the development of postoperative delirium and that Tau-based plasma proteins can serve as risk biomarkers of postoperative delirium in patients.

Increasing evidence suggests that plasma Tau-PT217 and Tau-PT181 are newly identified AD plasma biomarkers^{19-27, 38}. Previous research shows that plasma Tau-PT181 concentrations can distinguish amyloid β-positive MCI and AD patients (highest levels), Aβ-positive cognitively unimpaired older adults and MCI patients (intermediate levels), and Aβ-negative young adults and cognitively unimpaired older adults (lowest levels)²³. Moreover, plasma Tau-PT181 concentration distinguishes AD dementia from frontotemporal dementia, vascular dementia, progressive supranuclear palsy, corticobasal syndrome, Parkinson's disease, or multiple systems atrophy²³. Additional research suggests that changes in plasma levels of Tau-PT217 were associated with the changes in CSF levels of Tau-PT217²⁴ and the development of AD^{25,27}.

In the present study, we found both Tau-PT217 and Tau-PT181 were associated with the presence and severity (Table 2) of postoperative delirium. The findings indicate that patients with underlying AD neuropathogenesis are more likely to develop delirium. Moreover, it is important to perform better preoperative brain health assessments in patients.

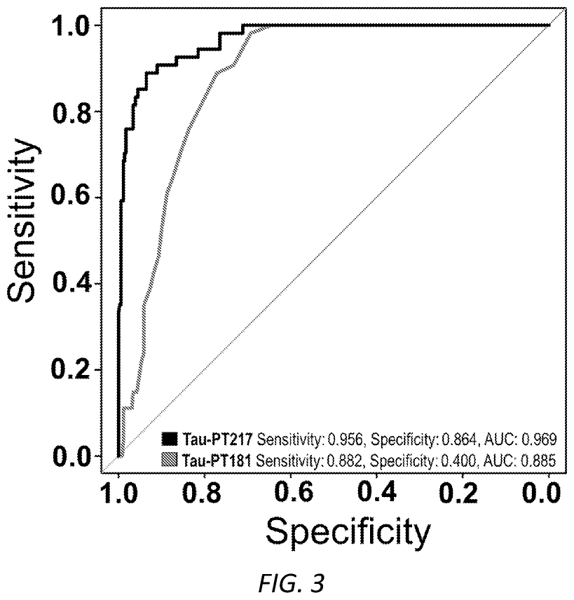

In the adjusted models, plasma Tau-PT217 (AUC 0.969) had an increased ability to discriminate delirious patients from non-delirious patients in comparison to Tau-PT181 (AUC 0.885; FIG. 3). These results were consistent with the previous findings that plasma Tau-PT217 has significantly higher accuracy than plasma Tau-PT181 in differentiating the neuropathologically defined AD from non-AD, clinical AD dementia versus other neurodegenerative diseases or among the PSEN1 mutation carriers versus PSEN1 mutation noncarriers^{25,27}.

Previous studies have demonstrated that the increase in plasma concentrations of Tau-PT217 occurs earlier than changes in positron emission tomography signal of Tau in cortex²⁶. Thus, the findings from present study specifically suggest that there is an association between pre-clinical AD neuropathogenesis (e.g., elevation in plasma Tau-PT217) and the development of postoperative delirium. Future research should evaluate whether the development of postoperative delirium could be a clinical manifestation of preclinical AD. This will take on increasing importance as effective treatments for AD become available, particularly if they need to be started in the pre-clinical phase to be most effective.

Plasma Tau-PT217 and Tau-PT181 are associated with the development of AD and long-term cognitive impairment¹⁹⁻²⁷. But whether plasma Tau-PT217 and Tau-PT181 can also serve as predictors of postoperative delirium was previously unknown. Therefore, the present study focused on assessing whether preoperative plasma Tau-PT217 and Tau-PT181 are associated with postoperative delirium. In future studies, we will use the established system to determine whether both preoperative and postoperative plasma Tau-PT217 and Tau-PT181 are associated with the development of other types of perioperative neurocognitive disorders, including delayed neurocognitive recovery and postoperative neurocognitive disorder.

The average Memorial Delirium Assessment Scale (MDAS) score of the participants in the present study was 6.6 (Table 1). Breitbart et al. stated that MDAS Scores ≥13 indicate the presence of delirium³⁶. However, the participants in the study by Breitbart et al. included psychiatry consult patients³⁶. On the other hand, Marcantonio et al. showed that the best MDAS cutoff for postoperative delirium was 5 in the participants with surgery for hip fracture repair³⁵. Therefore, an average MDAS score of 6.6 in present study is plausible though on the very mild side.

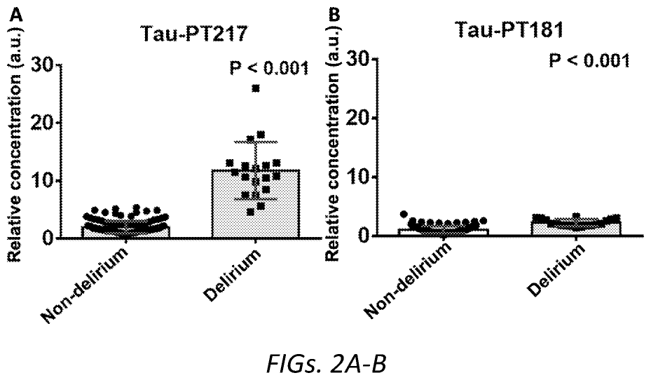

A strength of the present study includes the application of the novel Nanoneedle technology to detect phosphorylated Tau in blood samples (FIGS. 2A-B, 4A-B, and 5A-F). The Nanoneedle sensors have critical dimensions smaller than 100 nm, i.e., 50-500 times smaller than the bead-based detecting platforms. Each nanoneedle is a single molecule biosensor, functionalized with capture antibodies, allowing precise quantitation of analytes by digitally counting the number of nanoneedles with a positive signal. Compared to existing methods, nanoneedles require less sample volume (2-5 μl), have better sensitivity and lower per assay cost due to their scalable fabrication process.

The present data demonstrated that patients who developed postoperative delirium had higher preoperative plasma concentrations of Tau-PT217 or Tau-PT181 than those who did not develop postoperative delirium. Preoperative plasma concentration of Tau-PT217 or Tau-PT181 predicted the presence and severity of postoperative delirium, with Tau-PT217 being the most strongly associated with these outcomes. Thus, provided herein are methods that use preoperative plasma levels of one or both of Tau-PT217 and/or Tau-PT181 in a subject to predict whether the subject is likely to develop postoperative delirium. The methods can also include recommending or providing interventions to reduce risk or mitigate the severity or consequences of postoperative delirium.

### Methods of Determining Risk of Developing Postoperative Delirium (POD)

The methods described herein use preoperative plasma levels of one or both of Tau-PT217 and/or Tau-PT181 in a subject to predict whether the subject is likely to develop postoperative delirium (POD), e.g., has a risk of developing POD that is above the risk of a reference subject. The following is an exemplary sequence of the 441-amino acid tau protein (NCBI Reference Sequence: NP_005901.2, also known as microtubule-associated protein tau isoform 2 (MAPT2)):

| (SEQ ID NO: 1) | |
|---|---|
| 1 | maeprqefev medhagtygl gdrkdqggyt mhqdqegdtd aglkesplqt ptedgseepg |
| 61 | setsdakstp taedvtaplv degapgkqaa aqphteipeg ttaeeagigd tpsledeaag |
| 121 | hvtqarmvsk skdgtgsddk kakgadgktk iatprgaapp gqkgqanatr ipaktppapk |
| 181 | tppssgeppk sgdrsgyssp gspgtpgsrs rtpslptppt repkkvavvr tppkspssak |
| 241 | srlqtapvpm pdlknvkski gstenlkhqp gggkvqiink kldlsnvqsk cgskdnikhv |
| 301 | pgggsvqivy kpvdlskvts kcgslgnihh kpgggqvevk sekldfkdrv qskigsldni |
| 361 | thvpgggnkk iethkltfre nakaktdhga eivykspvvs gdtsprhlsn vsstgsidmv |
| 421 | dspqlatlad evsaslakqg l |

The present methods can be used in human subjects, e.g., elderly human subjects who are age 60 or above, or age 65 or above, who are planning to undergo a surgical procedure, e.g., a surgical procedure requiring general or regional anesthesia. In some embodiments, the subject is planning to undergo a surgical procedure in the next 24-48 hours, 3 days, 4 days, 7 days, month, or longer. Subjects who are “planning to” undergo a surgical procedure includes subjects who need surgery on an non-urgent basis, urgent basis, or subjects who are unable to make their own decisions with regard to planning surgery, including subjects whose health care providers recommend the surgical procedure. In some embodiments, the subjects have a normal preoperative cognitive function, e.g., Mini-Mental State Examination (MMSE) score more than 24, or a MMSE score that is normal for that individual.

The methods can include obtaining a sample from a subject, and evaluating the presence and/or level of Tau-PT217 and/or Tau-PT181 in the sample, and comparing the presence and/or level with one or more references, e.g., a control reference that represents a normal level of Tau-PT217 and/or Tau-PT181, e.g., a level in an unaffected subject, and/or a disease reference that represents a level of Tau-PT217 and/or Tau-PT181 associated with risk of developing POD, e.g., a level in a subject who has a risk of developing POD that is above that of the general population or above that of a cohort of subjects matched by age, gender, or other characteristic. Suitable reference values can include at least 1.75 or 2 times above a level in a cohort of control subjects who does not develop POD.

As used herein the term “sample”, when referring to the material to be tested for the presence of a biological marker using the method of the invention, includes inter alia whole blood, plasma, and serum.

The methods can include isolating and/or purifying Tau-PT217 and/or Tau-PT181 from the sample. Various methods are well known within the art for the identification and/or isolation and/or purification of Tau-PT217 and/or Tau-PT181 from a sample. An “isolated” or “purified” biological marker can be substantially free of cells, cellular material or other contaminants from the source from which the biological marker is derived; in some embodiments, a whole blood sample is centrifuged, e.g., at 500 g for 10 minutes, to remove cells and cellular debris, and plasma supernatant is collected.

The presence and/or level of Tau-PT217 and/or Tau-PT181 protein can be evaluated using high-sensitivity or ultrasensitive protein detection methods known in the art (i.e., an assay having a limit of detection under 1 picomolar (0.1 femtomoles in 100 ul). Examples include Meso Scale Discovery (MSD); Single-Molecule Arrays (SIMOA); droplet digital ELISA (ddELISA),⁴³ Molecular On-bead Signal Amplification for Individual Counting (MOSAIC),⁴⁴ Single-Molecule Counting (SMC); LUMINEX; SOMAscan Assays; mass spectrometry (e.g., MALDI-MS) and mass cytometry (e.g., CyTOF) (see, e.g., Cohen and Walt, Chem. Rev. 2019, 119, 293-321), or nanoneedles (see, e.g., WO2022047085, WO2020176793, WO2019051181). The methods can include the use of revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an enzyme linked immunosorbent assay (ELISA)-based method may be used, wherein a surface such as a bead, slide, or the wells of a mictrotiter plate, is coated with reagents, e.g., antibodies (i.e., capture antibodies) that bind to Tau-PT217 and/or Tau-PT181. The sample containing or suspected of containing the biological marker is then applied to the surface. After a sufficient amount of time, during which antibody-antigen complexes would have formed, the surface is washed to remove any unbound moieties, and a second molecule that can be used to detect the antibody-antigen complexes, e.g., a detection antibody that binds to the capture antibody or to the tau protein, e.g., a detectably labelled antibody, is added. Again, after a sufficient period of incubation, the surface is washed to remove any excess, unbound molecules, and the presence of the labeled molecule is determined using methods known in the art. In some embodiments, an ELISA method, e.g., SIMOA, MOSAIC, nanoneedles, or another ultrasensitive method, is used. SIMOA assays for Tau-PT217 or Tau-PT181 are described in ref⁵³. The detectably labelled antibody can include a tag, e.g., a label (detectable moiety) or a purification moiety, e.g., FLAG, hexahistidine (6-HIS), or hemagglutinin (HA). Examples of detectable substances for use as labels include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, or ³H.

Methods for making reagents, e.g., antibodies or antigen-binding fragments thereof, that bind to Tau-PT217 or Tau-PT181 are known in the art, and antibodies are commercially or publicly available. For example, anti-Tau-PT217 antibodies are commercially available from Affinity Biosciences; Biorbyt; Bioss Inc.; Eli Lilly; GeneTex; Novus Biologicals; Thermo Fisher Scientific; and United States Biological. Anti-Tau-PT181 antibodies are commercially available from Anogen; Antigenix America Inc.; BioLegend; Cell Signaling Technology; Creative Biolabs; Creative Diagnostics; dianova GmbH; MyBioSource.com; Thermo Fisher Scientific; and United States Biological. In some embodiments, these antibodies, or antigen-binding fragments thereof (such as Fv, Fab, Fab′, F(ab′)2), can be used in the assays described herein.

Classical immunoassays monitor a signal, e.g., an absorbance signal due to the increased amount of chromogen as the analyte concentration increases. Nanoneedle technology detects a spectrum shift from individual nanoneedles originating from additional mass deposition on each nanoneedle, forming an antibody-antigen sandwich complex.

In some embodiments, the capture antibody for Tau-PT217 is ThermoFisher Cat. No. 44-744, or IBA493 (Eli Lilly) or an antigen-binding fragment thereof (e.g., Fab2), and/or for Tau-PT181 is AT270 (ThermoFisher Cat No. MN1050) or ADx252 (ADx NeuroSciences) or an antigen-binding fragment thereof (e.g., Fab2). In some embodiments, the detection antibody is an anti-tau antibody, e.g., LRL or 4G10E2 (Eli Lilly and Company), Dx204 (ADx NeuroSciences), or Tau12 (Sigma Aldrich).

In some embodiments, mass spectrometry, and particularly matrix-assisted laser desorption/ionization mass spectrometry (MALDI-MS), surface-enhanced laser desorption/ionization mass spectrometry (SELDI-MS), or LC-MS, is useful for the detection of Tau-PT217 and/or Tau-PT181 (see, e.g., Barthélemeny et al., Alzheimers Res Ther. 2020 Mar. 17; 12(1):26 (quantitative mass spectrometry for detection of Tau-PT217 and/or Tau-PT181); Gobom et al., Mol Neurodegener. 2022 Dec. 12; 17(1):81 (LC-MS); U.S. Pat. Nos. 5,118,937; 5,045,694; 5,719,060; 6,225,047).

In some embodiments, the presence and/or level of Tau-PT217 and/or Tau-PT181 is comparable to the presence and/or level of Tau-PT217 and/or Tau-PT181 in the disease reference, then the subject can be identified as being at risk of developing POD. In some embodiments, the subject has no overt signs or symptoms of dementia or delirium, but the presence and/or level of Tau-PT217 and/or Tau-PT181 is comparable to the presence and/or level of Tau-PT217 and/or Tau-PT181 in the disease reference, then the subject has an increased risk of developing POD. In some embodiments, once it has been determined that a person has an increased risk of developing POD, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of Tau-PT217 and/or Tau-PT181, e.g., a control reference level that represents a normal level of Tau-PT217 and/or Tau-PT181, e.g., a level in a subject who is not at risk of developing POD (e.g., a control level determined from a cohort of subjects who do not develop POD), and/or a disease reference that represents a level of Tau-PT217 and/or Tau-PT181 associated with a cohort of subjects who develop POD.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not develop POD.

A disease reference subject is one who has (or has an increased risk of developing) one POD. An increased risk is defined as a risk above the risk of subjects in the general population.

Thus, in some cases the level of Tau-PT217 and/or Tau-PT181 in a subject being less than or equal to a reference level of Tau-PT217 and/or Tau-PT181 is indicative of a low or normal risk of developing POD). In other cases the level of Tau-PT217 and/or Tau-PT181 in a subject being greater than or equal to the reference level of Tau-PT217 and/or Tau-PT181 is indicative of an increased risk of developing POD. In some embodiments, the amount by which the level in the subject is the less than the reference level is sufficient to distinguish a subject from a control subject, and optionally is a statistically significantly less than the level in a control subject. In cases where the level of Tau-PT217 and/or Tau-PT181 in a subject being equal to the reference level of Tau-PT217 and/or Tau-PT181, the “being equal” refers to being approximately equal (e.g., not statistically different).

In some embodiments, the higher the level of Tau-PT217 and/or Tau-PT181 in a subject, the higher the risk of developing more severe POD. Thus the

The predetermined value can depend upon the particular population of subjects (e.g., human subjects) selected. For example, an apparently healthy population will have a different ‘normal’ range of levels of Tau-PT217 and/or Tau-PT181 than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

### Interventions

In some embodiments, once a subject has been identified as at risk of developing POD based on a method described herein, then an intervention can be applied. For example, preoperatively, the use of antihistamines, anticholinergics, tricyclic antidepressants, benzodiazepines, muscle relaxants, meperidine, gabapentinoids, and scopolamine can be avoided, as well as avoiding perioperative polypharmacy. Administration of prophylactic medications, including low-dose antipsychotics, e.g., olanzapine, quetiapine, or risperidone, can be included. Where subjects are undergoing sedation, alpha-2 agonists, e.g., clonidine or dexmedotomidine, can be used. During the surgery, an intravenous bolus, e.g., of 0.5 mg/kg, ketamine during anesthetic induction;⁴⁸ regional and neuraxial anaesthesia; xenon anesthesia; and administration of dexmedotomidine, can also be used during anesthesia to reduce risk of POD.⁴⁹ Pre- and post-operatively, behavioral interventions and additional care measures, including following comprehensive geriatric assessment (CGA)-based perioperative care, deliberately orienting the patient to place, time and reason for hospitalization; encouraging mobilization with at least daily walks; maintaining hydration (avoiding prolonged (>6 h) fluid fasting); avoiding sleep deprivation; reminding patients to use their glasses and hearing aids when appropriate; and encouraging family involvement, can be used to reduce risk of POD in subjects who are identified as being at risk using a method described herein.⁴⁹ For example, the ABCDE bundle⁵⁰ or Hospital Elder Life Program (HELP) or modified HELP⁵¹ can be used. See also, e.g., Inouye et al., N Engl J Med. 1999 Mar. 4; 340(9):669-76; Vidán et al., J Am Geriatr Soc. 2009 November; 57(11):2029-36. Pharmacologic interventions can include pre- and post-operative pain management (preferably with opioid-sparing analgesia, e.g., including the use of NSAIDs and paracetamol/acetaminophen); administration of melatonin receptor agonists such as melatonin and ramelteon; and administration of synthetic corticosteroids such as dexamethasone.⁵²

### EXAMPLES

The invention is further described in the following examples, which do not limit the scope of the invention described in the claims.

### Example 1. Preoperative Plasma Tau-PT217 and Tau-PT181 are Associated with Postoperative Delirium

### Methods

The following materials and methods were used in this example.

Study Enrollment. This prospective observational cohort study was performed at Massachusetts General Hospital, Boston, MA. The Mass General Brigham Institutional Review Board approved the study protocol. Patients were included if they were scheduled to have an elective knee replacement, hip replacement, or laminectomy under general or spinal anesthesia at the study hospital, were 65 years or older, and were proficient in English.

Subjects were excluded from participation if they had any of the following: (1) past medical history of neurological and psychiatric diseases including AD, other forms of dementia, stroke, or psychosis; (2) severe visual or hearing impairment; (3) were current smokers; or (4) taking antibiotics within one week of the day of surgery because disturbance of gut microbiota may affect brain function. Eligible patients were approached for participation by clinical research coordinators during preoperative clinic visits. Written informed consent was obtained at the time of enrollment, prior to initiation of the study procedures. There have been no significant changes in the surgery or anesthesia practice since the start of the study. The manuscript was written according to the STrengthening the Reporting of OBservational studies in Epidemiology (STROBE) criteria.

Anesthesia, Surgery, and Plasma Sample Collection. All participants received standardized perioperative care, including standard postoperative pain management (e.g., patient-controlled analgesia [PCA] with hydromorphone). Depth of sedation was at the discretion of the treating provider but was not captured in the current study.

Sample Preparation. Five ml of blood was collected from the participants before the anesthesia and surgery when the intravenous catheter was inserted. Blood samples were centrifuged at 500 g for 10 minutes, and plasma supernatant was collected. The plasma was collected in an EDTA tube and was immediately placed on ice. The plasma was then stored in a −80° C. degree freezer until the time of measurement when the samples were thawed. All of the measures in the current study were performed using the same methods with double-blind design.

Nanoneedle technology: More than 20,000 nanoneedles are integrated on a silicon substrate assigned to detect one analyte. Each nanoneedle is a single molecule biosensor functionalized with antibodies. Since phosphorylated Tau in blood samples are present at low abundance, the binding events of phosphorylated Tau on the nanoneedle sensor array follow Poisson statistics, i.e., one or no molecule is captured on each nanoneedle. This allows precise quantitation of analytes by digitally counting the number of nanoneedles that have a positive signal. In the present study, we developed sandwich-type Tau-PT217 and Tau-PT181 assays to measure the ultra-low concentration of protein levels in patient blood samples. The nanoneedle chip was provided by NanoMosaic (Woburn, MA, USA). Nanoneedles were fabricated in an array format with a spacing of 1.8 μm. Each nanoneedle had a diameter of less than 100 nm. The nanoneedle surfaces were modified with 0.5% 3-aminopropyltrimethoxysilane (APTMS) and activated with 2% glutaraldehyde. This enables antibodies to covalently bind on the surface of the nanoneedles⁴¹. We used 5 μg/ml capture antibody (anti-Tau 46 antibody (Cat #T9450, 1:1000; Sigma-Aldrich, St Louis, MO) concentration and incubated with chip overnight. We then washed the chip in PBS and incubated it with blocking solution for 1 hour. 5 μL of plasma samples were thawed and diluted 2× into the dilution buffer provided by NanoMosaic Inc. The diluted sample was incubated on the chip for 2 hours. After washing, a biotinylated detection antibody (ThermoFisher 44-744 for Tau-PT217 or ThermoFisher MN1050 for Tau-PT181) at 0.5 μg/ml was incubated on the chip. Next, 0.5 μg/ml streptavidin-HRP conjugate was incubated for 30 minutes, and 3,3′,5,5′-tetramethylbenzidine (TMB) provided by NanoMosaic was applied on the chip for 15 minutes. The enzymatic reaction produced a non-soluble precipitate on the nanoneedles when the sandwich complex was present. The precipitate changes the local refractive index of the nanoneedle and induces a color shift intrinsic to the nanoneedle property⁴². The nanoneedles are imaged under a dark field configuration with a CMOS color camera before and after the assay using the Tessie™ nanoneedle assay instrument from NanoMosaic (Woburn, MA, USA). Software provided by NanoMosaic (Woburn, MA, USA) analyzed the colors of all nanoneedles and reported the percentage of the color-shifted number of nanoneedles, which was used as the relative concentration with arbitrary unit (a.u.) in FIGS. 2A-B. We used phospho-specific antibodies to Tau-PT217 (ThermoFisher 44-744) and Tau-PT181 (ThermoFisher MN1050) as the detection antibodies for Tau-PT217 and Tau-PT181 measurement. The specificity of the phospho-specific antibodies to Tau-PT217 and Tau-PT181 were validated with peptide array, described in the following section.

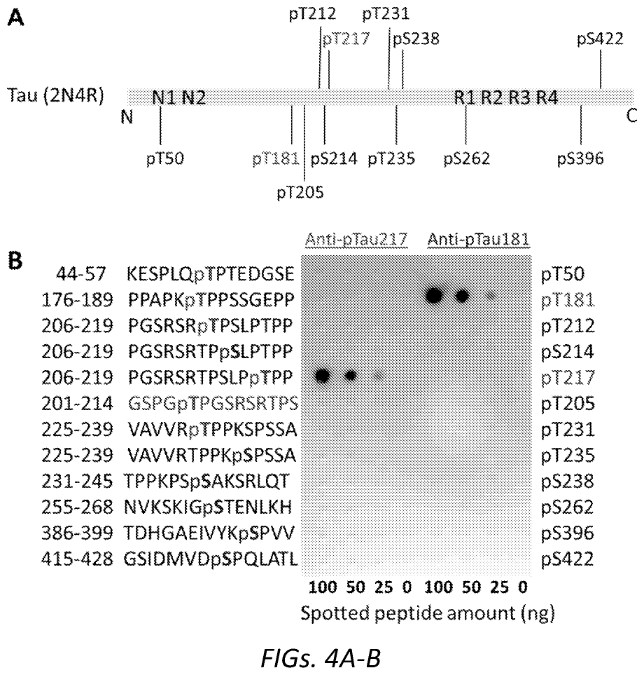

Peptide synthesis, purification, and analysis: Peptides (FIG. 4B) were synthesized on an automated robotic peptide synthesizer (Intavis, Model Multipep) using Standard Fmoc solid-phase chemistry. Phospho amino acids were incorporated using Fmoc-Phospho-Ser(OBzl)-OH and Fmoc-Phospho-Thr(OBzl)-OH. Preloaded Wang resins were used for the respective C-terminal amino acids. Subsequent amino acids were coupled using optimized (to generate peptides containing more than 90% of the desired full-length peptides) cycles consisting of Fmoc removal (deprotection) with 25% piperidine in NMP followed by coupling of Fmoc-AAs using HBTU/HOBt/DIEA activation. Each deprotection or coupling was followed by several washes of the resin with DMF to remove excess reagents. After the peptides were assembled and the final Fmoc group removed, peptide resins were washed with dimethylformamide, dichloromethane, and methanol three times each and air-dried. Peptides were cleaved from the solid support and deprotected using an odor-free cocktail (TFA/triisopropyl silane/water/DODT; 94/2.5/2.5/1.0 v/v) for 2.5 h at room temperature. Peptides were precipitated using cold methyl tertiary butyl ether (MTBE). The precipitate was washed 2 times in MTBE, dissolved in a solvent (0.1% trifluoroacetic acid in 30% Acetonitrile/70% water), followed by freeze-drying. Purifications were performed using preparative reverse-phase HPLC. Peptides were characterized by Microbore HPLC and Matrix-Assisted Laser Desorption/Ionization Mass Spectrometry (MALDI-MS).

Validation of antibody specificity with peptide array: We used a peptide array to validate the specificity of the antibodies used in this study. As outlined in FIGS. 4A-B, phosphorylated peptides of 14 amino acids at known phosphorylated sites were synthesized. The sequences of these peptides is as follows; pT indicates the phosphorylated Tyrosine residue, pS is phosphoserine:

| Position | Sequence | SEQ ID NO: |
|---|---|---|
| 44-57 | KESPLQpTPTEDGSE | 1. |
| 176-189 | PPAPKpTPPSSGEPP | 2. |
| 206-219 | PGSRSRpTPSLPTPP | 3. |
| 206-219 | PGSRSRTPpSLPTPP | 4. |
| 206-219 | PGSRSRTPSLPpTPP | 5. |
| 201-214 | GSPGpTPGSRSRTPS | 6. |
| 225-239 | VAVVRpTPPKSPSSA | 7. |
| 225-239 | VAVVRTPPKpSPSSA | 8. |
| 231-245 | TPPKPSpSAKSRLQT | 9. |
| 255-268 | NVKSKIGpSTENLKH | 10. |
| 386-399 | TDHGAEIVYKpSPVV | 11. |
| 415-428 | GSIDMVDpSPQLATL | 12. |

We applied the Bio-Dot system (Bio-Rad, #1706545) to test Tau-PT217 (ThermoFisher 44-744) and Tau-PT181 (ThermoFisher MN1050) antibodies against the 12 peptides phosphorylated at different sites. First, we used 100 μl TBS (Tris Buffered Saline, Bio-Rad, #1706435) per well to prewet nitrocellulose membrane prior to placing it in the apparatus. Then, we rehydrated the membrane to ensure uniform binding of the designed peptides. The flow valve was adjusted for the vacuum chamber opening to air, and wells were loaded with 100 ng, 50 ng, 25 ng and 0 ng peptides. After the peptide solution passed through the membrane thoroughly, we used 200 μl TTBS (Tween 20, Tris Buffered Saline) to wash the membrane twice. We then added 300 μl of the blocking solution to each well. After allowing gravity filtration to occur until the blocking solution was wholly drained from each well, we then repeated the wash step twice with TTBS solution. 100 μl of anti-Tau-PT217 or anti-Tau-PT181 primary antibody solutions (0.5 μg/mL, ThermoFisher #44-744 or ThermoFisher #MN1050) were added to each sample well. After allowing gravity filtration to occur until the antibody solution completely drained from the sample wells, we then applied the vacuum to remove any excess liquid. After washing 3 times, vacuum was used until the wash solution was drained from the wells. 100 μl of secondary antibody (Rabbit anti-Mouse IgG (H+L) Secondary Antibody, HRP, #61-6520, 1:5000 diluted; Thermo-fisher) was added, drained, and washed for 3 times. The membrane was developed with chemiluminescent solution (Pierce™ ECL Western Blotting Substrate, 32106, Thermo-fisher) and imaged with Bio-Rad ChemiDoc™ MP Imaging System. As shown in FIG. 4B, titrated signals were observed for both antibodies to their specific residues. No cross-reactive signals were observed to other residues on the dot blot, thus supporting the conclusion that the measurement was specific to Tau-PT217 and Tau-PT181.

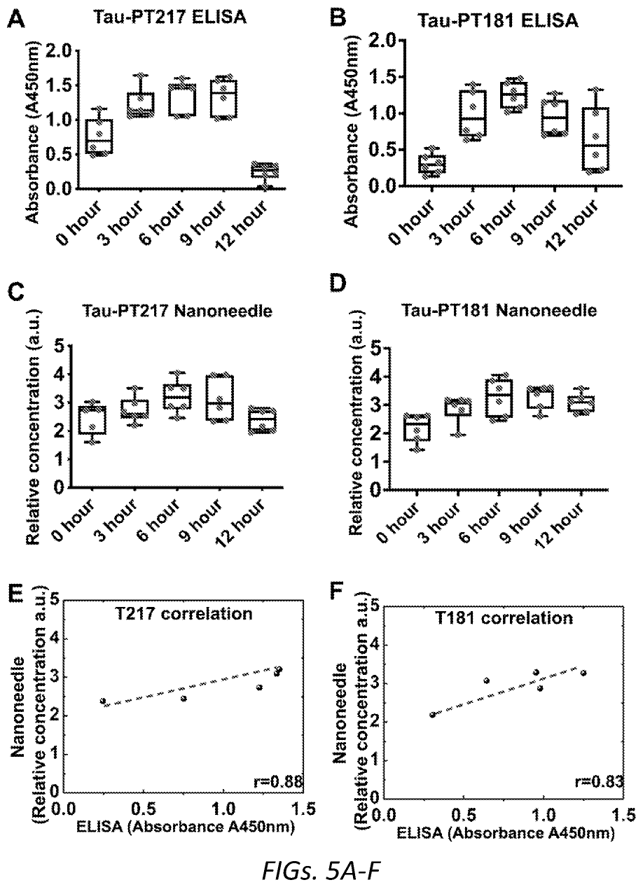

Validation of nanoneedle method with ELISA: Measurement from the Nanoneedle technology was compared with the gold standard ELISA method in cell line samples. Human SH-SY5Y cells were treated with 3% anesthetic sevoflurane for 0, 3, 6, 9 and 12 hours to induce the pathogenesis of Tau-PT217 and Tau-PT181. Phospho-Tau (Thr181) ELISA kit (Cat #58537, Cell Signaling Technology, Danvers, MA) and Phospho-Tau (Thr217) ELISA kit (Cat #58672, Cell Signaling Technology) were used to measure Tau-PT181 and Tau-PT217 levels in the post-treated SH-SY5Y cells. 50 μL cell lysate sample was loaded to each well on the ELISA plate. Cell lysates after 0, 3, 6, 9 and 12 hours were each measured for 6 times. Absorbance was determined by using spectrophotometer (model) at 450 nm. The Nanoneedle technology was used to measure Tau-PT217 and Tau-PT181 from the same batch of SY5Y cell samples using the methods as described in the previous section. Compared to the ELISA measurement, the cell samples were further diluted by 10× into the cell extraction buffer before loading onto the Nanoneedle chip. 10 μL sample volume was loaded to each well on the Nanoneedle chip. This was to ensure the Nanoneedle signals from the current experiment are in the same range as those in the human plasma measurements in present study, in order to validate the Nanoneedle technology at the same concentration range. As can be seen in FIGS. 5A-D, both ELISA and Nanoneedle measurement demonstrated time-dependent changes in the amounts of human Tau-PT217 and Tau-PT181 in the SY5Y cells. Specifically, the sevoflurane treatment increased the amounts of human Tau-PT217 and Tau-PT181 up to 9 hours after the treatment. The reductions in the amounts of human Tau-PT217 and Tau-PT181 at 12 hours could be due to cell death after 12 hours of sevoflurane treatment, thus fewer cells were collected for the measurement. The Pearson's correlation coefficients between Nanoneedle and ELISA measurements are 0.88 and 0.83 for Tau-PT217 and Tau-PT181, respectively (FIGS. 5E-F). These data indicated that the Nanoneedle methods and ELISA methods were well correlated. Furthermore, Nanoneedle technology was able to quantitate protein levels with 50× less protein amounts when compared to the ELISA method.

Exposures—Measurement of Tau-PT217 and Tau-PT181 in Plasma by Nanoneedle. The primary exposures of interest were the measurements of Tau-PT217 and Tau-PT181 from preoperative blood samples. The measurement of the phosphorylated Tau was performed blinded to postoperative delirium status to avoid bias. As Tau-PT217 and Tau-PT181 present at an ultra-low abundance level not detectable with traditional western blot or enzyme-linked immunosorbent assays (ELISA) in blood, we developed in-house phosphorylated Tau assays using the Nanoneedle technology to measure preoperative plasma concentrations of Tau-PT217 and Tau-PT181. The protein levels are reported in relative units specific to the Nanoneedle technology (i.e., relative concentration with arbitrary unit) since Tau-PT217 or Tau-PT181 recombinant protein standards are not available at present. Therefore, the term plasma concentration used throughout the manuscript refers to relative concentration but not absolute concentration of Tau-PT217 or Tau-PT181 in the plasma of participants. All samples from the participants were randomly assigned to different Nanoneedle batches during the measurement of Tau-PT217 or Tau-PT181. The assays of Tau-PT217 and Tau-PT181 were performed in triplicate with 5 ul in each well of the batch. The average intra-assay coefficient of variations (CV) of plasma Tau-PT217 and Tau-PT181 in the postoperative delirium group were 39.9% and 21.0%, respectively. But the difference among the values of plasma Tau-PT217 and Tau-PT181 obtained in the three measurements was not significant.

All measurement results were above the lower limit of detection of the Nanoneedle assay. Assay specificity was validated with dot blot measurement of the phospho-specific antibodies against an array of synthesized peptides phosphorylated at different residues along the full-length Tau protein (FIGS. 4A-B). Accuracy of the Nanoneedle method was validated by comparing measurements of Tau-PT217 and Tau-PT181 in cell culture samples using gold standard ELISA method (FIGS. 5A-F).

Outcomes. Trained clinical research coordinators interviewed participants on postoperative day 1 and/or 2. Confusion Assessment Measurement (CAM) is a diagnostic algorithm used to determine the presence or absence of delirium, which has high reliability^{34,35}. One hundred of the 139 participants had the CAM on both days, and 3 of the 18 participants with postoperative delirium and 36 of the 121 participants without postoperative delirium had the CAM on one day. Delirium was assessed using the CAM once per day between 8:00 am and 12:00 noon. The primary outcome was the presence of postoperative delirium defined based on CAM performance on either postoperative day 1 or postoperative day 2.

The secondary outcome was the severity of postoperative delirium, represented by the Memorial Delirium Assessment Scale (MDAS)^{35,36}, which quantifies symptoms related to delirium based on 10 features. Each feature is scored from 0 (best) to 3 (worst symptom) with a maximal score of 30. MDAS scores were evaluated for all patients, regardless of whether they met CAM criteria for delirium on that day. In the present study, the diagnosis of delirium presence was based on the results from CAM. The MDAS score was used to assess the severity of delirium independent of the results obtained from CAM. Postoperative Mini-Mental State Examination (MMSE) was performed as part of CAM^{34,37} and also for MDAS calculation on postoperative day 1 and/or day 2³⁷.

Statistical Power Calculation. We calculated, based on estimates from our previous study¹⁶, and performed in the design phase of the study, that a sample size of 130 participants would be sufficient to determine a Pearson correlation >0.20 between plasma Tau concentrations and MDAS delirium severity scores, assuming a two-sided hypothesis test with 80% power and 5% type I error.

Statistical Analysis. Descriptive statistics were conducted using methods appropriate for the variables under study. Means and standard deviations were used for continuously scaled variables that were normally distributed. Medians and 25^{th} and 75^{th} percentiles were used for skewed or ordinal data. Frequency counts and percentages or proportions were used for categorical variables. Differences in baseline characteristics between those who did and did not develop delirium were assessed with a t-test, Mann Whitney U-test (for non-normal continuous data), chi-square or Fisher's exact test (in the case of small cell counts), as appropriate. Because each plasma sample was measured in triplicate, generalized estimating equations were used to model variability across measurements. For this model, the subject was the clustering variable. The delirium outcome was specified as a binomial variable with a logit link. An independent covariance structure was assumed. Estimation was conducted using a robust (i.e., sandwich) estimation approach. Results are presented as odds ratio (OR) per one unit change in the biomarker and their associated 95% confidence intervals (CI). Youden's Index was utilized in order to evaluate the biomarker cutoff that best predicted the presence of delirium. Results are presented as the area under the receiver operating characteristic curve (AUC) for that Tau-PT217 and Tau-PT181 cutoff, sensitivity, and specificity. The AUC was calculated by computing the numeric value of the area under the ROC curve using the trapezoidal rule to assess the probability that the model would score a randomly drawn positive sample higher than a randomly drawn negative sample. The association between the biomarkers and delirium severity was estimated using a similar approach to delirium presence but with a Gaussian distribution and identity link. Results are presented as the beta coefficient (β) per unit change in Tau-PT217 or Tau-PT181 value and its associated 95% CI. Models were created to adjust the associations between the biomarkers and outcomes for age, education, and preoperative MMSE for both the primary and secondary outcomes. Variables for adjustment were based on previous studies as deemed clinically relevant. The assumptions for each model were considered by examining the residuals and calibration curves to ensure the models adequately fit the data. All analyses were conducted using R4.0.5 statistical software (Vienna, Austria). Where appropriate, all analyses use two-tailed hypothesis testing, with statistical significance interpreted at p<0.05.

### Results

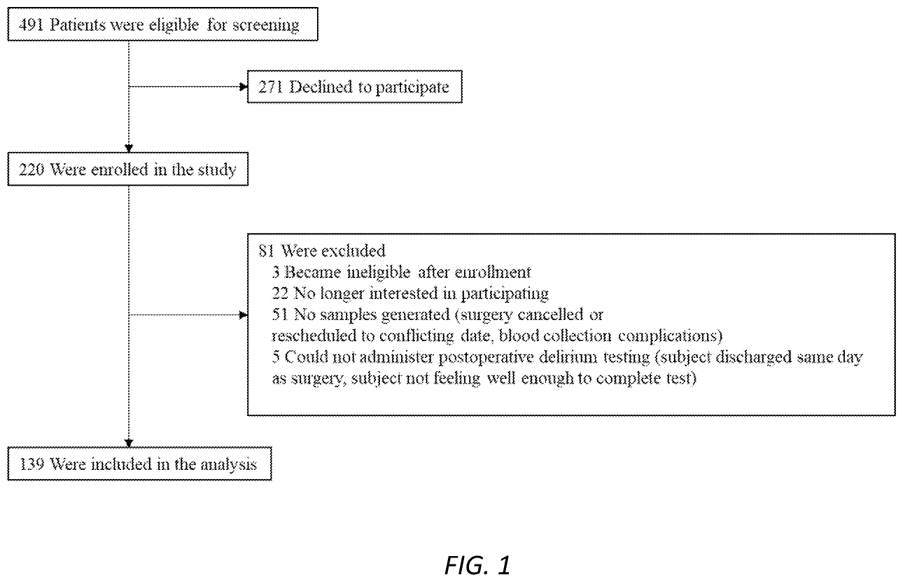

A total of 491 patients were screened, of which 220 participants were enrolled between November 2016 and February 2020. Eighty-one participants were excluded due to becoming ineligible after enrollment (N=3), no longer expressing interest in participating (N=22), having no plasma samples collected (N=51, surgery being canceled or rescheduled to conflicting date, plasma collection complications), or having no assessment of postoperative delirium on any of two days owing to participants being discharged the same day of surgery or not feeling well enough to complete testing (N=5). Thus, 139 participants were included in the final data analysis (FIG. 1). There were no significant complications among participants during the immediate postoperative period.

The baseline demographic and clinical characteristics of the 139 participants are presented in Table 1. There were no significant differences in age, gender, ethnicity, surgery type, anesthesia type, or preoperative Mini-Mental State Examination (MMSE) score between the participants with postoperative delirium (N=18) and those without postoperative delirium (N=121). The participants who developed postoperative delirium had less education years and lower postoperative MMSE score than the participants who did not develop postoperative delirium.

Each of the plasma samples was randomly assigned to different batches of the Nanoneedle measurement for Tau-PT217 and Tau-PT181. The measurement was performed in triplicate by using Nanoneedle technology. Before the measurement, specificity of antibody used in the assay was tested using peptide array (FIGS. 4A-B) and accuracy of the Nanoneedle technology was validated against the gold standard ELISA method (FIGS. 5A-F). In the present study, there was no significant difference among the values of plasma Tau-PT217 (F=0.530, P=0.589, one-way analysis of variance [ANOVA]) and Tau-PT181 (F=0.022, P=0.978, one-way ANOVA) obtained in the three different measurements.

The participants who developed postoperative delirium had higher preoperative plasma concentrations of Tau-PT217 (11.77+4.97 arbitrary unit [a.u.] versus 1.97+1.08 a.u., P<0.001) and Tau-PT181 (2.25+0.61 a.u. versus 1.06+0.63 a.u., P<0.001) than participants who did not develop the postoperative delirium (Table 1 and FIGS. 2A-B).

Preoperative Plasma Concentrations of Tau-PT217 and Tau-PT181 were Associated with Postoperative Delirium.

Results of the unadjusted analyses and multivariable models are presented in Table 2. After adjustment for age, education, and preoperative MMSE, the preoperative plasma concentrations of Tau-PT217 (OR 2.05, 95% CI. 1.61-2.62, P<0.001) and Tau-PT181 (OR 4.12, 95% CI: 2.55-6.67, P<0.001) were associated with postoperative delirium in separate models. Preoperative plasma concentrations of Tau-PT217 and Tau-PT181 had high sensitivity (0.956 and 0.882) and variable specificity (0.864 and 0.400) for predicting postoperative delirium (FIG. 3). The AUC for Tau-PT217 and Tau-PT181 were 0.969 and 0.885, respectively (FIG. 3).

Preoperative Plasma Concentrations of Tau-PT217 and Tau-PT181 were Associated with the Severity of Postoperative Delirium.

In the crude models, increased preoperative plasma concentrations of Tau-PT217 or Tau-PT181 were associated with an increase in postoperative delirium severity. This association persisted after adjustment for age, education, and preoperative MMSE (P coefficient: 0.14, 95% CI: 0.09-0.19, P<0.001 for Tau-PT217 and 0.41, 95% CI: 0.12-0.70, P=0.006 for Tau-PT181; Table 2).

TABLE 1
Demographic Characteristics and Plasma Concentrations
of Phosphorylated Tau in the Participants

| | | | No | |
|---|---|---|---|---|
| | Total | Delirium, | Delirium, | |
| | N = 139 | N = 18 | N = 121 | p |
| Age, mean ± SD | 73 ± 6 | 73 ± 5 | 73 ± 6 | 0.828 |
| Female, n (%) | 76 (55) | 9 (50) | 67 (55) | 0.561 |
| Non-White or | 8 (6) | 2 (11) | 6 (5) | 0.277 |
| Hispanic, n (%) | | | | |
| Educational | 16.36 ± 2.39 | 15.5 ± 1.51 | 16.29 ± 2.91 | 0.024 |
| attainment, years, | | | | |
| mean ± SD | | | | |
| Surgery type, n (%) | | | | |
| Knee replacement | 96 (69) | 13 (72) | 83 (69) | 0.907 |
| Hip replacement | 33 (24) | 3 (17) | 30 (25) | |
| Spinal stenosis | 10 (7) | 2 (11) | 8 (7) | |
| Anesthesia type, n (%) | | | | |
| General | 62 (45) | 7 (39) | 55 (45) | 0.800 |
| Spinal | 77 (55) | 11 (61) | 66 (55) | |
| MMSE, mean ± SD | | | | |
| Preanesthesia/ | 29.11 ± 1.03 | 28.94 ± 1.11 | 29.13 ± 1.02 | 0.223 |
| surgery score | | | | |
| Postanesthesia/ | 28.76 ± 1.42 | 27.56 ± 2.00 | 28.94 ± 1.23 | 0.001 |
| surgery score | | | | |
| Peak score of MDAS, | 2.96 ± 2.20 | 6.61 ± 2.03 | 2.41 ± 1.64 | <0.001 |
| mean ± SD | | | | |
| Tau-PT217 | 3.23 ± 4.32 | 11.77 ± 4.97 | 1.97 ± 1.08 | <0.001 |
| (arbitrary unit) | | | | |
| Tau-PT181 | 1.20 ± 0.89 | 2.25 ± 0.61 | 1.06 ± 0.63 | <0.001 |
| (arbitrary unit) | | | | |

TABLE 2
Tau-PT217 and Tau-PT181 were significant predictors
of postoperative delirium incidence and severity.

| | The Incidence of Postoperative Delirium | | | |
|---|---|---|---|---|
| | | | Adjusted for Age | |
| | Unadjusted | | and Preoperative MMSE | |
| | Odds Ratio | | Odds Ratio | |
| | (95% CI) | P-Value | (95% CI) | P-Value |
| Tau-PT217 | 1.93 (1.59, 2.35) | <0.001 | 2.05 (1.61, 2.62) | <0.001 |
| Tau-PT181 | 3.65 (2.25, 5.92) | <0.001 | 4.12 (2.55, 6.67) | <0.001 |

The Severity of Postoperative Delirium

| | | | Adjusted for Age | |
|---|---|---|---|---|
| | Unadjusted | | and Preoperative MMSE | |
| | β Coefficient | | β Coefficient | |
| | (95% CI) | P-Value | (95% CI) | P-Value |
| Tau-PT217 | 0.14 (0.09, 0.19) | <0.001 | 0.14 (0.09, 0.19) | <0.001 |
| Tau-PT181 | 0.39 (0.10, 0.68) | 0.009 | 0.41 (0.12, 0.70) | 0.006 |

Tau-PT217, Tau phosphorylation at threonine 217;
Tau-PT181, Tau phosphorylation at threonine 181;
CI, confidence interval.

### REFERENCES

1. Vutskits L, Xie Z. Lasting impact of general anaesthesia on the brain: mechanisms and relevance. Nat Rev Neurosci 2016; 17(11):705-717.
2. Inouye S K. Delirium in older persons. N Engl J Med 2006; 354(11):1157-65.
3. Rudolph J L, Jones R N, Rasmussen L S, et al. Independent vascular and cognitive risk factors for postoperative delirium. Am J Med 2007; 120(9):807-13.
4. Witlox J, Eurelings L S, de Jonghe J F, et al. Delirium in elderly patients and the risk of postdischarge mortality, institutionalization, and dementia: a meta-analysis. JAMA 2010; 304(4):443-51.
5. Martin B J, Buth K J, Arora R C, et al. Delirium as a predictor of sepsis in post-coronary artery bypass grafting patients: a retrospective cohort study. Crit Care 2010; 14(5):R171.
6. Koster S, Hensens A G, van der Palen J. The long-term cognitive and functional outcomes of postoperative delirium after cardiac surgery. Ann Thorac Surg 2009; 87(5):1469-74.
7. Marcantonio E R, Flacker J M, Michaels M, et al. Delirium is independently associated with poor functional recovery after hip fracture. J Am Geriatr Soc 2000; 48(6):618-24.
8. Saczynski J S, Marcantonio E R, Quach L, et al. Cognitive trajectories after postoperative delirium. N Engl J Med 2012; 367(1):30-9.
9. Marcantonio E R, Goldman L, Mangione C M, et al. A clinical prediction rule for delirium after elective noncardiac surgery. JAMA 1994; 271(2):134-9.
10. Robinson T N, Raeburn C D, Tran Z V, et al. Postoperative delirium in the elderly: risk factors and outcomes. Ann Surg 2009; 249(1):173-8.
11. Gou R Y, Hshieh T T, Marcantonio E R, et al. One-Year Medicare Costs Associated With Delirium in Older Patients Undergoing Major Elective Surgery. JAMA Surg 2021; 156(5):430-442.
12. Davis D H, Muniz Terrera G, Keage H, et al. Delirium is a strong risk factor for dementia in the oldest-old: a population-based cohort study. Brain 2012; 135(Pt 9):2809-16.
13. Ags/Nia Delirium Conference Writing Group P C, Faculty. The American Geriatrics Society/National Institute on Aging Bedside-to-Bench Conference: Research Agenda on Delirium in Older Adults. J Am Geriatr Soc 2015; 63(5):843-52.
14. Fong T G, Davis D, Growdon M E, et al. The interface between delirium and dementia in elderly adults. Lancet Neurol 2015; 14(8):823-832.
15. Fong T G, Jones R N, Shi P, et al. Delirium accelerates cognitive decline in Alzheimer disease. Neurology 2009; 72(18):1570-5.
16. Xie Z, Swain C A, Ward S A, et al. Preoperative cerebrospinal fluid beta-Amyloid/Tau ratio and postoperative delirium. Ann Clin Transl Neurol 2014; 1(5):319-328.
17. Wang Y, Mandelkow E. Tau in physiology and pathology. Nat Rev Neurosci 2016; 17(1):5-21.
18. Holtzman D M, Carrillo M C, Hendrix J A, et al. Tau: From research to clinical development. Alzheimers Dement 2016; 12(10):1033-1039.
19. Tatebe H, Kasai T, Ohmichi T, et al. Quantification of plasma phosphorylated tau to use as a biomarker for brain Alzheimer pathology: pilot case-control studies including patients with Alzheimer's disease and down syndrome. Mol Neurodegener 2017; 12(1):63.
20. Mielke M M, Hagen C E, Xu J, et al. Plasma phospho-tau181 increases with Alzheimer's disease clinical severity and is associated with tau- and amyloid-positron emission tomography. Alzheimers Dement 2018; 14(8):989-997.
21. Janelidze S, Mattsson N, Palmqvist S, et al. Plasma P-tau181 in Alzheimer's disease: relationship to other biomarkers, differential diagnosis, neuropathology and longitudinal progression to Alzheimer's dementia. Nat Med 2020; 26(3):379-386.
22. Thijssen E H, La Joie R, Wolf A, et al. Diagnostic value of plasma phosphorylated tau181 in Alzheimer's disease and frontotemporal lobar degeneration. Nat Med 2020; 26(3):387-397.
23. Karikari T K, Pascoal T A, Ashton N J, et al. Blood phosphorylated tau 181 as a biomarker for Alzheimer's disease: a diagnostic performance and prediction modelling study using data from four prospective cohorts. Lancet Neurol 2020; 19(5):422-433.
24. Palmqvist S, Insel P S, Stomrud E, et al. Cerebrospinal fluid and plasma biomarker trajectories with increasing amyloid deposition in Alzheimer's disease. EMBO Mol Med 2019; 11(12):e11170.
25. Palmqvist S, Janelidze S, Quiroz Y T, et al. Discriminative Accuracy of Plasma Phospho-tau217 for Alzheimer Disease vs Other Neurodegenerative Disorders. JAMA 2020; 324(8):772-781.
26. Janelidze S, Berron D, Smith R, et al. Associations of Plasma Phospho-Tau217 Levels With Tau Positron Emission Tomography in Early Alzheimer Disease. JAMA Neurol 2021; 78(2):149-156.
27. Thijssen E H, La Joie R, Strom A, et al. Plasma phosphorylated tau 217 and phosphorylated tau 181 as biomarkers in Alzheimer's disease and frontotemporal lobar degeneration: a retrospective diagnostic performance study. Lancet Neurol 2021; 20(9):739-752.
28. Vasunilashorn S M, Ngo L H, Dillon S T, et al. Plasma and cerebrospinal fluid inflammation and the blood-brain barrier in older surgical patients: the Role of Inflammation after Surgery for Elders (RISE) study. J Neuroinflammation 2021; 18(1):103.
29. Vasunilashorn S M, Ngo L H, Inouye S K, et al. Apolipoprotein E genotype and the association between C-reactive protein and postoperative delirium: Importance of gene-protein interactions. Alzheimers Dement 2020; 16(3):572-580.
30. Fong T G, Vasunilashorn S M, Ngo L, et al. Association of Plasma Neurofilament Light with Postoperative Delirium. Ann Neurol 2020; 88(5):984-994.
31. Vasunilashorn S M, Dillon S T, Chan N Y, et al. Proteome-Wide Analysis using SOMAscan Identifies and Validates Chitinase-3-Like Protein 1 as a Risk and Disease Marker of Delirium Among Older Adults Undergoing Major Elective Surgery. J Gerontol A Biol Sci Med Sci 2021.
32. Tripp B A, Dillon S T, Yuan M, et al. Targeted metabolomics analysis of postoperative delirium. Sci Rep 2021; 11(1):1521.
33. Ballweg T, White M, Parker M, et al. Association between plasma tau and postoperative delirium incidence and severity: a prospective observational study. Br J Anaesth 2021; 126(2):458-466.
34. Inouye S K, van Dyck C H, Alessi C A, et al. Clarifying confusion: the confusion assessment method. A new method for detection of delirium. Ann Intern Med 1990; 113(12):941-8.
35. Marcantonio E, Ta T, Duthie E, et al. Delirium severity and psychomotor types: their relationship with outcomes after hip fracture repair. J Am Geriatr Soc 2002; 50(5):850-7.
36. Breitbart W, Rosenfeld B, Roth A, et al. The Memorial Delirium Assessment Scale. J Pain Symptom Manage 1997; 13(3):128-37.
37. Simon S E, Bergmann M A, Jones R N, et al. Reliability of a structured assessment for nonclinicians to detect delirium among new admissions to postacute care. J Am Med Dir Assoc 2006; 7(7):412-5.
38. Yang C C, Chiu M J, Chen T F, et al. Assay of Plasma Phosphorylated Tau Protein (Threonine 181) and Total Tau Protein in Early-Stage Alzheimer's Disease. J Alzheimers Dis 2018; 61(4):1323-1332.
39. Mei X, Zheng H L, Li C, et al. The Effects of Propofol and Sevoflurane on Postoperative Delirium in Older Patients: A Randomized Clinical Trial Study. J Alzheimers Dis 2020; 76(4):1627-1636.
40. Shi Z, Mei X, Li C, et al. Postoperative Delirium Is Associated with Long-term Decline in Activities of Daily Living. Anesthesiology 2019; 131(3):492-500.
41. Liang F, Clarke N, Patel P, et al. Scalable photonic crystal chips for high sensitivity protein detection. Opt Express 2013; 21(26):32306-12.
42. Liang F, Zhang Y, Hong W, et al. Direct Tracking of Amyloid and Tu Dynamics in Neuroblastoma Cells Using Nanoplasmonic Fiber Tip Probes. Nano Lett 2016; 16(7):3989-94.
43. Cohen L, Cui N, Cai Y, Garden P M, Li X, Weitz D A, Walt D R. Single Molecule Protein Detection with Attomolar Sensitivity Using Droplet Digital Enzyme-Linked Immunosorbent Assay. ACS Nano. 2020; 14(8):9491-501. doi: 10.1021/acsnano.0c02378.
44. Wu C, Dougan T J, Walt D R. MOSAIC: High-Throughput, High-Multiplex Digital Protein Detection with Attomolar Sensitivity. Wu, C., Dougan, T. J. & Walt, D. R. High-Throughput, High-Multiplex Digital Protein Detection with Attomolar Sensitivity. ACS Nano, doi:10.1021/acsnano.1c08675 (2022).
45. Barthélemeny et al., Cerebrospinal fluid phospho-tau T217 outperforms T181 as a biomarker for the differential diagnosis of Alzheimer's disease and PET amyloid-positive patient identification. Alzheimers Res Ther. 2020 Mar. 17; 12(1):26.
46. Gobom et al., Antibody-free measurement of cerebrospinal fluid tau phosphorylation across the Alzheimer's disease continuum. Mol Neurodegener. 2022 Dec. 12; 17(1):81.
47. Whitlock et al., Postoperative Delirium. Minerva Anestesiol. 2011 April; 77(4): 448-456.
48. Hudetz J A, Patterson K M, Iqbal Z, Gandhi S D, Byrne A J, Hudetz A G, et al. Ketamine attenuates delirium after cardiac surgery with cardiopulmonary bypass. J Cardiothorac Vasc Anesth. 2009; 23(5):651-657.
49. Janjua M S, Spurling B C, Arthur M E. Postoperative Delirium. [Updated 2022 May 15]. In: StatPearls [Internet]. Treasure Island (FL): StatPearls Publishing; 2022 January-. Available from: ncbi.nlm.nih.gov/books/NBK534831/
50. Marra A, Ely E W, Pandharipande P P, Patel M B. The ABCDEF Bundle in Critical Care. Crit Care Clin. 2017 April; 33(2):225-243.
51. Chen C C, Li H C, Liang J T, Lai I R, Purnomo J D T, Yang Y T, Lin B R, Huang J, Yang C Y, Tien Y W, Chen C N, Lin M T, Huang G H, Inouye S K. Effect of a Modified Hospital Elder Life Program on Delirium and Length of Hospital Stay in Patients Undergoing Abdominal Surgery: A Cluster Randomized Clinical Trial. JAMA Surg. 2017 Sep. 1; 152(9):827-834.
52. Jin Z, Hu J, Ma D. Postoperative delirium: perioperative assessment, risk reduction, and management. Br J Anaesth. 2020 October; 125(4):492-504
53. Bayoumy S, Verberk I M W, den Dulk B, Hussainali Z, Zwan M, van der Flier W M, Ashton N J, Zetterberg H, Blennow K, Vanbrabant J, Stoops E, Vanmechelen E, Dage J L, Teunissen C E. Clinical and analytical comparison of six Simoa assays for plasma P-tau isoforms P-tau181, P-tau217, and P-tau231. Alzheimers Res Ther. 2021 Dec. 4; 13(1):198.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining risk of developing postoperative delirium (POD) in a subject, preferably wherein the subject is a human over the age of 60, the method comprising:
optionally obtaining a sample comprising plasma from the subject;
determining a level of Tau isoforms phosphorylated at threonine 217 (Tau-PT217) and/or a level of Tau isoforms phosphorylated at threonine 181 (Tau-PT181) in the sample;
comparing the levels of Tau-PT217 and/or Tau-PT181 in the sample to reference levels of Tau-PT217 and/or Tau-PT181; and
identifying a subject who has a level of Tau-PT217 and/or Tau-PT181 above the reference level as being at risk of developing postoperative delirium (POD).

2. The method of claim 1, wherein the subject has no clinical signs of Alzheimer's disease (AD), AD Related Dementias (ADRD), and delirium.

3. The method of claim 1, wherein the subject has a preoperative Mini-Mental State Examination (MMSE) score more than 24.

4. The method of claim 1, wherein determining levels of Tau-PT217 and/or Tau-PT181 in the sample comprises using an ultrasensitive assay.

5. The method of claim 4, wherein the ultrasensitive assay comprises a nanoneedle-based assay, Single-Molecule Arrays (SIMOA); Molecular On-bead Signal Amplification for Individual Counting (MOSAIC); Meso Scale Discovery (MSD); Single-Molecule Counting (SMC); nucleic acid linked immune-sandwich assay (NULISA); LUMINEX; SOMAscan Assays; mass spectrometry (e.g., MALDI-MS), and/or mass cytometry (e.g., CyTOF).

6. A method of reducing risk of developing postoperative delirium (POD) in a subject, the method comprising:
optionally obtaining a sample comprising plasma from the subject;
determining a level of Tau isoforms phosphorylated at threonine 217 (Tau-PT217) and/or a level of Tau isoforms phosphorylated at threonine 181 (Tau-PT181) in the sample;
comparing the levels of Tau-PT217 and/or Tau-PT181 in the sample to reference levels of Tau-PT217 and/or Tau-PT181; and
identifying a subject who has a level of Tau-PT217 and/or Tau-PT181 above the reference level as being at risk of developing POD; and
administering an intervention to reduce the risk of developing POD to the subject.

7. The method of claim 6, wherein the subject has no clinical signs of Alzheimer's disease (AD), AD Related Dementias (ADRD), and delirium.

8. The method of claim 6, wherein the subject has a preoperative Mini-Mental State Examination (MMSE) score more than 24.

9. The method of claim 6, wherein determining levels of Tau-PT217 and/or Tau-PT181 in the sample comprises using an ultrasensitive assay.

10. The method of claim 9, wherein the ultrasensitive assay comprises a nanoneedle-based assay, Single-Molecule Arrays (SIMOA); Molecular On-bead Signal Amplification for Individual Counting (MOSAIC); Meso Scale Discovery (MSD); Single-Molecule Counting (SMC); nucleic acid linked immune-sandwich assay (NULISA); LUMINEX; SOMAscan Assays; mass spectrometry (e.g., MALDI-MS), and/or mass cytometry (e.g., CyTOF).

11. The method of claim 6, wherein the intervention comprises avoiding the use of antihistamines, anticholinergics, tricyclic antidepressants, benzodiazepines, muscle relaxants, meperidine, gabapentinoids, and scopolamine; and avoiding perioperative polypharmacy.

12. The method of claim 6, wherein the intervention comprises administration of prophylactic low-dose antipsychotics, optionally olanzapine, quetiapine, or risperidone, can be included.

13. The method of claim 6, wherein the intervention comprises sedation using alpha-2 agonists, optionally clonidine or dexmedotomidine.

14. The method of claim 6, wherein the intervention comprises administering ketamine during anesthetic induction; use of regional and neuraxial anaesthesia; xenon anesthesia; and/or administration of dexmedotomidine.

15. The method of claim 6, wherein the intervention comprises pre- and post-operative behavioral interventions and additional care measures, optionally following comprehensive geriatric assessment (CGA)-based perioperative care, ABCDE bundle, or Hospital Elder Life Program (HELP) or modified HELP.

16. The method of claim 15, wherein the behavioral interventions and additional care measures comprise deliberately orienting the patient to place, time and reason for hospitalization; encouraging mobilization with at least daily walks; maintaining hydration (avoiding prolonged (>6 h) fluid fasting); avoiding sleep deprivation; and reminding patients to use their glasses and hearing aids when appropriate.

17. The method of claim 6, wherein the intervention comprises pharmacologic interventions, optionally selected from pre- and post-operative pain management (optionally with opioid-sparing analgesia); administration of NSAIDs and paracetamol/acetaminophen; administration of melatonin receptor agonists such as melatonin and ramelteon; and administration of synthetic corticosteroids such as dexamethasone.
